(19) United States
(12) Patent Application Publication
(10) Pub. No.: US 2023/0293159 A1
(43) Pub. Date: Sep. 21, 2023

(54) ENDOVASCULAR INJECTABLE STENTS FOR CARDIOVASCULAR DRUG DELIVERY

(71) Applicant: Massachusetts Institute of Technology, Cambridge, MA (US)

(72) Inventors: Sahab BABAEE, Arlington, MA (US); Mazen ALBAGHDADI, Cambridge, MA (US); Mohammed CHOWDHURY, Cambridge, MA (US); Carlo TRAVERSO, Newton, MA (US)

(21) Appl. No.: 18/010,428

(22) PCT Filed: Jun. 21, 2021
(86) PCT No.: PCT/US2021/038283

§ 371 Date: Dec. 14, 2022

Related U.S. Application Data
(60) Provisional application No. 63/041,556, filed on Jun. 19, 2020.

(51) Int. Cl. A61B 17/00

(52) U.S. Cl. CPC A61B 17/00234; A61B 2018/00571

(57) ABSTRACT

Herein is described a stent platform capable of intravascular local delivery of therapeutics through multipoint injection of drug agents into the vascular walls and endocardial surfaces. More specifically, an endovascular injectable stent made of kirigami skin is wrapped around a soft linear actuator and used for intravascular local drug delivery of therapeutics. This platform addresses an unmet need of treating aortic stenosis and atherosclerotic cardiovascular disease.

### CROSS-REFERENCES TO RELATED APPLICATIONS

The present application is based on, claims priority to, and incorporates herein by reference in its entirety, U.S. Provisional Patent Application No. 63/041,556 filed Jun. 19, 2020, entitled “Endovascular Injectable Stents for Cardiovascular Drug Delivery.”

### STATEMENT REGARDING FEDERALLY SPONSORED RESEARCH

Not applicable.

### BACKGROUND

Valvular aortic stenosis (“AS”) leads to progressive narrowing of the aortic valve orifice resulting in endocarditis, embolic events, arrhythmias, heart failure and sudden death. The burden of valvular heart disease in a growing population of individuals above the age of 75 is considerable with more than 1 in 8 people having moderate or severe aortic stenosis. In adult patients with symptomatic severe AS who do not undergo valve replacement, nearly 75% will not be alive at 5 years following symptom onset. The only available treatment for patients with AS is transcatheter or surgical valve replacement—a costly procedure associated with significant healthcare costs. Unfortunately, at present there is no medical therapy available to treat or prevent AS. Thus, there is a pressing unmet need to develop drug delivery therapies to prevent progression of diseases within the vasculature, such as aortic stenosis.

Drug-coated balloons (“DCBs”) and drug-eluting stents (“DES”) significantly improve endovascular revascularization outcomes by reducing restenosis in patients with obstructive atherosclerotic coronary and peripheral artery disease as compared to percutaneous transluminal angioplasty (“PTA”). Restenosis is associated with recurrence of patient's symptoms and 25-35% of successfully treated atherosclerotic lesions restenose within 3-6 months generating increased costs for additional revascularization procedures, atherectomy or bypass surgery. Thus, restenosis resulting in arterial re-narrowing following DCB or DES remains a primary limitation of endovascular revascularization. One of the principal mechanisms of restenosis risk following treatment with endovascular revascularization devices, DCBs in particular, is the inefficient delivery of anti-proliferative drugs, such as paclitaxel (“PTX”), to the arterial wall. Most DCBs are coated with 3 ug/mm² paclitaxel. Usually, 60-90 seconds is used for balloon inflation, allowing for a homogenous transfer of only about 8-18% of the drug to the treated vessel wall. In addition, injury to the arterial wall that subsequently triggers neointimal hyperplasia during endovascular interventions such as stenting or DCB angioplasty may hamper the ability of anti-proliferatives delivered from these devices to prevent restenosis. Thus, there is a pressing need for improved methods of arterial drug delivery that maximize drug delivery and minimize arterial injury to reduce restenosis following endovascular revascularization.

### SUMMARY

Herein is described a stent platform capable of intravascular local delivery of therapeutics through multipoint injection of drug agents into tubular structures of the body, such as the vascular walls. More specifically, an endovascular injectable stent made of kirigami skin is wrapped around a soft linear actuator and used for intravascular local drug delivery of therapeutics. This platform addresses an unmet need of treating aortic stenosis and atherosclerotic cardiovascular disease, among others. The stent platform comprises a stretchable snakeskin-inspired kirigami shell integrated with a pneumatic linear soft actuator with the capacity to deposit drug depots circumferentially in the vasculature, valvular, myocardial, and endocardial regions.

According to some aspects of the present disclosure, a stent for treating tissue within tubular structures of a subject is provided. The stent includes a tubular body extending along a central axis and configured to move between a retracted position and an elongated position, and a plurality of projections formed into the tubular body, in which each projection forms a cutting edge to pierce a tissue within a tubular structure of the subject. Each projection among the plurality of projections is configured to change orientation relative to the central axis when the tubular body moves between the retracted position and the elongated position.

According to some aspects of the present disclosure, a stent system for treating a tissue of a subject is provided. The system includes a tubular body extending along a central axis to form a lumen within the tubular body, an actuator received within the lumen and configured to move the tubular body between a retracted position and an elongated position, and a pattern of a plurality of cuts formed along the tubular body and extending through the tubular body to the lumen. The pattern of the plurality of cuts deploy into a plurality of interconnected projections that are configured to extend radially away from the tubular body relative to the central axis to engage a tissue within a tubular structure of a subject when the tubular body is moved towards the elongated position.

According to some aspects of the present disclosure, a method of inserting a stent into a tubular structure a subject is provided. The method includes positioning a stent to a target tissue site, the stent having a tubular body extending along a central axis to form a lumen within the tubular body and pressurizing an actuator received within the lumen to move the tubular body from a retracted position to an elongated position. A surface of the tubular body includes a pattern of a plurality of cuts configured to deploy into a plurality of interconnected projections as the tubular body is moved into the elongated position to engage the target tissue site of the subject.

The foregoing and other aspects and advantages of the disclosure will appear from the following description. In the description, reference is made to the accompanying drawings which form a part hereof, and in which there is shown by way of illustration a preferred configuration of the disclosure. Such configuration does not necessarily represent the full scope of the disclosure, however, and reference is made therefore to the claims and herein for interpreting the scope of the disclosure.

### BRIEF DESCRIPTION OF THE DRAWINGS

The invention will be better understood and features, aspects and advantages other than those set forth above will become apparent when consideration is given to the following detailed description thereof. Such detailed description makes reference to the following drawings.

FIG. 1 illustrates a perspective view of a kirigami-inspired stent in an extended position according to one aspect of the present disclosure.

FIG. 2 illustrates a perspective view, including a cut-away, of the kirigami-inspired stent of FIG. 1 in a retracted position.

FIG. 3 illustrates a perspective detail view of denticle-like projection elements of the kirigami-inspired stent of FIG. 1 in a deployed position.

FIG. 4 illustrates a plan view of a pattern of cuts forming the denticle-like projection elements of the kirigami-inspired stent of FIG. 2 in a stowed position.

FIG. 5 illustrates a perspective exploded view of an actuator for the kirigami-inspired stent of FIG. 2.

FIG. 6 illustrates a perspective detail view of a fiber reinforcement for the actuator of FIG. 5.

FIG. 7 illustrates a perspective view of another non-limiting example of a kirigami-inspired stent in an extended position according to another aspect of the present disclosure.

FIG. 8 illustrates a schematic of a penetration depth of a projection element of the kirigami-inspired stents.

FIG. 9 illustrates a schematic of an exemplary cut forming the projection elements of the kirigami-inspired stent of FIG. 1.

FIG. 10 illustrates a schematic of an exemplary cut forming the projection elements akin to the kirigami-inspired stent of FIG. 7.

FIG. 11 illustrates a schematic of another exemplary cut forming the projection elements akin to the kirigami-inspired stent of FIG. 7.

FIG. 12 illustrates a schematic of yet another exemplary cut forming the projection elements akin to the kirigami-inspired stent of FIG. 7.

FIG. 13 illustrates a perspective view of another non-limiting example of a kirigami-inspired stent in a retracted position according to another aspect of the present disclosure.

FIG. 14 illustrates a schematic of the projection elements of the kirigami-inspired stent of FIG. 13 including etched striations according to one aspect of the present disclosure.

FIG. 15 illustrates an exemplary method of loading a therapeutic agent onto a kirigami-inspired stent to produce a drug eluting stent.

FIG. 16 illustrates a flow diagram of a method of inserting and removing a kirigami-inspired stent according to one aspect of the present disclosure.

FIG. 17 illustrates an exemplary schematic of a kirigami-inspired stent inserted into a subject's vasculature.

FIG. 18 illustrates a perspective view of a molding process for making a cast or injection molded actuator body.

FIG. 19 illustrates a perspective view of the cast or injection molded actuator body of FIG. 18 out of the mold.

FIG. 20 illustrates a perspective view of the cast or injection molded actuator body of FIG. 19 with a fiber reinforcement wrapping.

FIG. 21 illustrates a perspective view of a cutting process for making an outer shell of a kirigami-inspired spent and a photograph of the result of the cutting process.

FIG. 22 illustrates a flow diagram of a surface treatment process for the outer shell of FIG. 21 and a photograph of the result of the surface treatment process.

FIG. 23 illustrates a perspective view of the outer shell of FIG. 21 in an assembled state in preparation for receiving an actuator.

FIG. 24 illustrates a photograph of a kirigami-inspired stent prior-to (left) and after (center and right) of a coating process.

FIG. 25 illustrates images of an exemplary spray coating apparatus to apply coatings onto a kirigami-inspired stent.

FIG. 26 illustrates images of an exemplary method of continuous microfluidic drug-PLGA droplet generation.

FIG. 27 illustrates a photograph of percutaneous delivery of the kirigami-inspired stent into an iliac artery during balloon occlusion of the descending aorta via contralateral femoral access.

FIG. 28 illustrates a photograph of intraoperative exposure and visualization of the iliac artery following percutaneous delivery.

FIG. 29 illustrates a photograph of gross examination of luminal surface of iliac artery demonstrating tissue marking dye in the pattern consistent with delivery from projection elements of the kirigami-inspired stent.

FIG. 30 illustrates a representative histologic assessment of tissue shown in FIG. 29 demonstrating penetration of the kirigami skin to 200 microns.

### DETAILED DESCRIPTION

Before any aspects of the invention are explained in detail, it is to be understood that the invention is not limited in its application to the details of construction and the arrangement of components set forth in the following description or illustrated in the following drawings. The invention is capable of other aspects and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The use herein of the term “axial” and variations thereof refers to a direction that extends generally along an axis of symmetry, a central axis, an axis of rotation, or an elongate direction of a particular component or system. For example, axially extending features of a component may be features that extend generally along a direction that is parallel to an axis of symmetry or an elongate direction of that component. Further, for example, axially aligned components may be configured so that their axes of rotation are aligned. Similarly, the use herein of the term “radial” and variations thereof refers to directions that are generally perpendicular to a corresponding axial direction. For example, a radially extending structure of a component may generally extend at least partly along a direction that is perpendicular to a longitudinal or central axis of that component. The use herein of the term “circumferential” and variations thereof refers to a direction that extends generally around a circumference of an object or around an axis of symmetry, an axis of rotation, a central axis, or an elongate direction of a particular component or system.

As also used herein, unless specified or limited otherwise, the terms “approximately” and “substantially” and variations thereof, when used relative to a numerical value, define a range of values within 20% of the numerical value (e.g., within 15%, 10%, or within 5%).

In some implementations, devices or systems disclosed herein can be utilized, manufactured, or treated using methods embodying aspects of the invention. Correspondingly, any description herein of particular features, capabilities, or intended purposes of a device or system is generally intended to include disclosure of a method of using such devices for the intended purposes, of a method of otherwise implementing such capabilities, of a method of manufacturing relevant components of such a device or system (or the device or system as a whole), and of a method of installing or utilizing disclosed (or otherwise known) components to support such purposes or capabilities. Similarly, unless otherwise indicated or limited, discussion herein of any method of manufacturing or using for a particular device or system, including installing the device or system, is intended to inherently include disclosure, as embodiments of the invention, of the utilized features and implemented capabilities of such device or system.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

The stent platform comprises a stretchable snakeskin-inspired kirigami shell integrated with a pneumatic linear soft actuator with the capacity to deposit drug depots circumferentially in tubular structures of a subject, including in the vasculature, valvular, myocardial, and endocardial regions. Such systems can be applied for a range of diseases affecting the cardiovascular system such as aortic stenosis and atherosclerotic cardiovascular diseases.

As explained herein, inspired from the skin of scaly-skin animals like snakes and sharks, an injectable stent was developed which is composed of a periodic array of denticle-like needles (e.g., a kirigami cylindrical shell) integrated with a linear actuator (e.g., a pneumatic soft actuator). The injectable stents can be manufactured in multiple length scales that can be easily deployed in the tubular lumen of a subject, such as arteries and other vasculature. By pressurizing the soft actuator, the kirigami needles buckle out (e.g., extend) such that the resulting needles provide required stiffness and radial expansion to enable injections of drug depots into the tissue of a subject (e.g., into tissues of the vasculature). These kirigami-based injectable stents serve as a class of drug-eluting stents, capable of releasing drug depots through multi-point deposition of drug particles, thereby enhancing sustained local delivery of therapeutics. These stents can be used to treat vascular disorders, including autoimmune, atherosclerotic, fibrotic, dysplastic, and congenital disorders, among others.

### Kirigami-Inspired Stent Examples

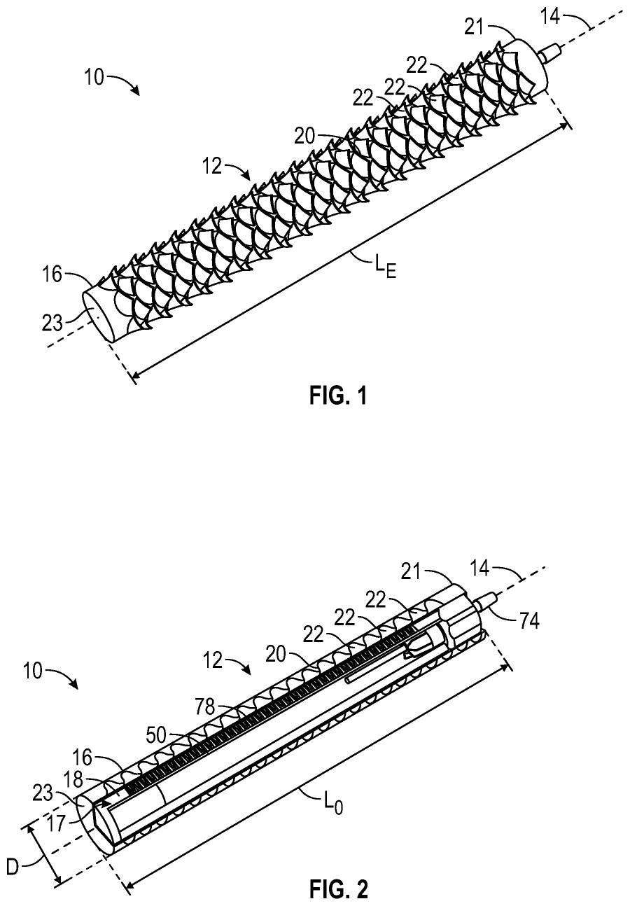

Referring to FIGS. 1 and 2, one non-limiting example of a stent 10 is illustrated. The stent 10 can define a tubular body 12 extending axially along a central axis 14 and configured for insertion into tubular structures of a subject's body, such as the vasculature regions, among the other regions previously described. The tubular body 12 of the stent 10 is configured to undergo a shape change in at least one dimension. In the illustrated non-limiting example, the tubular body 12 is axially extendable between a first, retracted position (FIG. 2) and a second, extended position (FIG. 1). In the extended position, the tubular body 12 is elongated in the axial direction relative to the retracted position. As will be described, the elongation of the tubular body 12 between the retracted position and the extended position is configured to deploy projections configured to pierce or engage tissue of a subject.

The tubular body 12 can include a cylindrical outer shell 16 forming a lumen 17 (e.g., a hollow core) and an actuator 18 arranged within the lumen 17 of the outer shell 16. The outer shell 16 can include at least one cut 20. In the illustrated non-limiting example, the outer shell 16 can include a patterned array of a plurality of interconnected cuts 20 (e.g., openings). In the illustrated non-limiting example, the plurality of cuts 20 extend along at least a portion of the axial length of the tubular body 12. For example, the plurality of cuts 20 can extend along at least 50% of an entire length Lo of the tubular body 12. According to some non-limiting examples, the plurality of cuts 20 can extend along between about 50% and about 100% of the entire length Lo of the tubular body 12. According to the illustrated non-limiting example, the plurality of cuts 20 can extend along between about 80% and about 95% of the entire length Lo of the tubular body 12. In the illustrated non-limiting example, the plurality of cuts 20 extend along at least a portion of the circumference of the tubular body 12. For example, the plurality of cuts 20 can extend along at least 50% of the circumference of the tubular body 12. According to some non-limiting examples, the plurality of cuts 20 can extend along between about 50% and about 100% of the circumference of the tubular body 12. According to the illustrated non-limiting example, the plurality of cuts 20 can extend along between about 90% and about 100% of the circumference of the tubular body 12.

The length L₀ of the tubular body 12 can be defined as an initial length between a first end 21 and an opposing send end 23 of the tubular body 12 when the tubular body 12 is in the retracted position (FIG. 2). According to some non-limiting examples, the length L₀ can be between about 0.1 cm and about 40 cm. According to other non-limiting examples, the length L₀ can be between about 1 cm and about 20 cm. According to yet further non-limiting examples, the length L₀ can be between about 1 cm and about 10 cm.

The tubular body 12 can also define a nominal outer diameter D, defined as an initial diameter of the outer shell 16 when the tubular body 12 is in the retracted position (FIG. 2). According to some non-limiting examples, the diameter D can be between about 1 mm and about 100 mm. According to other non-limiting examples, the diameter D can be between about 1 mm and about 50 mm. According to yet further non-limiting examples, the diameter D can be between about 1 mm and about 25 mm.

When the tubular body 12 is elongated from the retracted position to the extended position, the tubular body 12 can define an elongated length L_{E} (FIG. 1) that is greater relative to the initial length L₀. According to some non-limiting examples, the elongated length L_{E} can be between about 1% and about 100% greater than the initial length L₀. According to other non-limiting examples, the elongated length L_{E} can be between about 10% and about 80% greater than the initial length L₀. According to yet further non-limiting examples, the elongated length L_{E} can be between about 15% and about 40% greater than the initial length L₀.

The plurality of cuts 20 can be configured to form a kirigami-inspired pattern configured to undergo a shape change when a stress is axially applied along the outer shell 16. via the actuator 18. The at least one cut 20 can form at least one projection element 22. In the illustrated non-limiting example, the series of patterned cuts 20 can form a plurality of projection elements 22 (e.g., needles). When the tubular body 12 of the stent 10 is in a retracted position (FIG. 2), the projection elements 22 are substantially planar with the outer shell 16 and undeformed. When the tubular body 12 of the stent 10 is elongated (e.g., via the actuator 18) from the retracted position towards the extended position (FIG. 1), the projection elements 22 become deformed and deploy to extend radially outward from the outer shell (e.g., relative to the central axis 14).

For example, as will be described, the outer shell 16 can be configured to automatically respond to strain applied in a direction along the central axis 14. That is, the series of patterned cuts 20 form a surface on the outer shell 16 that buckles in response to applied axial strain to form a plurality of projection elements from that cut surface. In the illustrated non-limiting examples, the actuator 18 is configured to apply the axial strain, and that axial strain results in stress within the outer shell 16 that causes the projection elements 22 to extend outwards from an orientation in which the projection elements form a substantially uniform (e.g., flat) cylindrical surface, into an orientation in which the projection elements deploy radially outwards relative to the central axis 14. According to some non-limiting examples, the magnitude of applied axial strain to the outer shell 16 can correspond to a magnitude of radial extension of the projection elements 22. That is, owing to the pattern of cuts 20 formed in the outer shell 16, a surface is provided that transforms in a radial direction in response to strain applied in an axial direction.

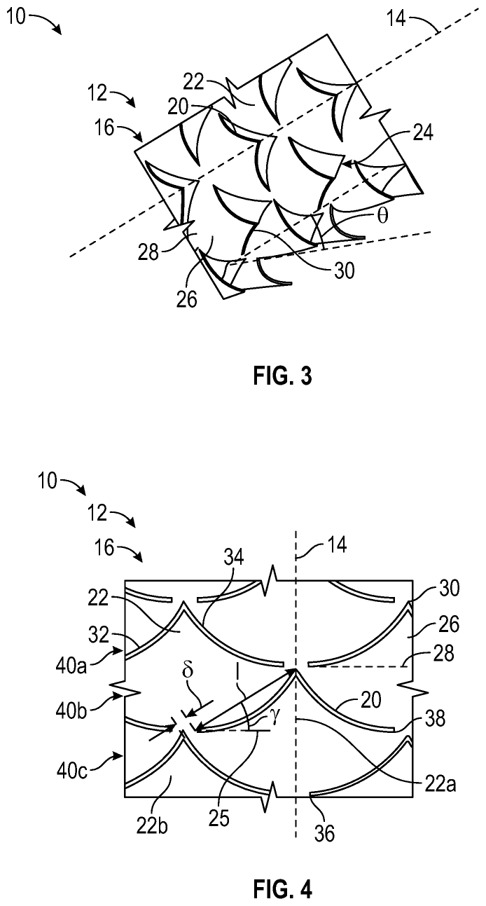

Referring now to FIGS. 3 and 4, with the tubular body 12 in an extended position, the projection elements 22 can deploy from an undeformed state (FIG. 4) to a deformed state (FIG. 3). In a deformed state, the projection elements 22 form denticle-like needles configured to pierce the tissue of a subject. According to some non-limiting examples, the projection elements 22 can be configured to piece the tissue without perforation of the tissue walls. This can be beneficial, for example, in delicate walls within the vasculature of a subject where preventing perforation can be vital (e.g., piercing into the tissue, without breaking through the entire thickness of the tissue walls). In the illustrated non-limiting example, the projection elements 22 define a convex three-dimensional surface forming a barb shaped needle. The projection elements 22, when deployed to the deformed state, reveal a plurality of openings 24 in the outer shell 16. The plurality of openings 24 extend through the outer shell 16 and into the lumen 17.

With particular reference to FIG. 3, with the projection elements 22 in a deployed position (with the tubular body 12 in the extended position), the protruding projection elements 22 can provide radial expansion up to 80% of the stent diameter (e.g., up to 60%, 40%, etc.). In the illustrated non-limiting example, the projection elements 22 can define a needle angle θ. The needle angle θ can be defined as the angle of a surface 26 of the projection element 22, formed between a base 28 and a needle tip 30, relative to the central axis 14 of the tubular body. According to some non-limiting examples, the projection elements 22 can define a needle angle θ between about 1 degree and about 90 degrees. According to other non-limiting examples, the projection elements 22 can define a needle angle θ between about 5 degrees and about 60 degrees. According to yet further non-limiting examples, the projection elements 22 can define a needle angle θ between about 10 degrees and about 40 degrees.

Referring now to FIG. 4, illustrating the projection elements 22 in an undeformed state (e.g., in a stowed state, with the tubular body in the retracted position), the cuts 20 can be configured as a pattern of denticle-like cuts. For example, each individual projection element 22 among the plurality of projection elements 22 formed by the pattern of cuts 20 can define a triangular shaped cutting edge, with first and second edges 32, 34 of the triangular shape being formed by a continuous cut 20, and the base 28 (illustrated in FIG. 4 as a broken line) being formed by an uncut portion. In the illustrated non-limiting, the projection elements 22 define a circular triangle. That is, the first and second edges 32, 34 of each projection element 22 define an arcuate shape. In the illustrated embodiment, the first and second edges 32, 34 of the projection element 22 define a convex arcuate shape. The arcuate shape of the first and second edges 32, 34 can define a radius of curvature between being a straight line and about a 100 mm radius. According to some non-limiting examples, the radius of curvature can be between about 1 mm and about 60 mm. According to other non-limiting examples, the radius of curvature can be between about 1 mm and about 40 mm. According to yet further non-limiting examples, the radius of curvature can be between about 1 mm and about 20 mm.

The patterned cuts 20 forming the projection elements 22 can be characterized by a needle length l, hinge length δ, and cut angle γ. The needle length l can be described as a characteristic length of the patterned cut 20 and can be considered as a length of the needle formed by the projection element 22. The needle length l can be defined by a distance between the needle tip 30 of the projection element 22 and either one of a first distal end 36 of the first edge 32 or a second distal end 38 of the second edge 34 (i.e., distal ends of the cut 20). According to some non-limiting examples, the projection elements 22 can define a needle length l between about 1 mm and about 60 mm. According to other non-limiting examples, the projection elements 22 can define a needle length l between about 1 mm and about 40 mm. According to yet further non-limiting examples, the projection elements 22 can define a needle length l between about 1 mm and about 20 mm.

The hinge length δ can be described as the width of ligaments forming an interstitial spacing separating adjacent cuts 20. The hinge length δ can be defined by a distance between the needle tip 30 of a first projection element 22a and either one of the first distal end 36 or the second distal end 38 of a second, adjacent projection element 22b. According to some non-limiting examples, the cuts 20 can define a hinge length δ between about 0.1 mm and about 10 mm. According to other non-limiting examples, the cuts 20 can define a hinge length δ between about 0.1 mm and about 5 mm. According to yet further non-limiting examples, the cuts 20 can define a hinge length δ between about 0.1 mm and about 2 mm.

The cut angle γ can be described as the angle of the cut 20 forming either one of the first and second edges 32, 34 of the projection element 22 relative to a plane 25 intersecting and orthogonal to the central axis 14. According to some non-limiting examples, the cuts 20 can define a cut angle γ between about 5 degrees and about 45 degrees. According to other non-limiting examples, the cuts 20 can define a cut angle γ between about 10 degrees and about 45 degrees. According to yet further non-limiting examples, the cuts 20 can define a cut angle γ between about 20 degrees and about 45 degrees. According to the illustrated non-limiting example, the cuts 20 define a cut angle γ of about 30 degrees.

Referring still to FIG. 4, a dimensionless ratio δ/l can be defined for a given pattern of cuts 20, the dimensionless ratio δ/l can correlate to a magnitude of pop-out deformation (e.g., a magnitude of needle angle θ, a magnitude of convex surface deformation in the projection elements 22, etc.) upon elongation of the tubular body 12. According to some non-limiting examples, the cuts 20 can define a dimensionless ratio δ/l between 0 and 1. According to other non-limiting examples, the cuts 20 can define a dimensionless ratio δ/l between 0 and about 0.5. According to yet further non-limiting examples, the cuts 20 can define a dimensionless ratio δ/l between 0 and about 0.2. According to the illustrated non-limiting example, the cuts 20 define a dimensionless ratio δ/l of about 0.13.

The cuts 20 forming the projection elements 22 can be evenly (e.g., periodically) circumferentially spaced around the outer shell 16 (see, e.g., FIG. 1). According to the illustrated non-limiting example, a plurality of rows of circumferentially spaced cuts 20 are arranged along the axial length of the outer shell 16. As best illustrated in FIG. 4, a first row 40a of circumferentially spaced cuts 20 can be rotationally offset from a second, adjacent row 40b of circumferentially spaced cuts 20. In the illustrated non-limiting example, the rotational offset between adjacent rows 40a, 40b of circumferentially spaced cuts 20 can be such that a needle tip 30 of a projection element 22 within the second row 40b is in rotational alignment between distal ends 36, 38 of two adjacent projection elements 22 within the first row 40a. That is, the rotational offset between adjacent rows 40a, 40b can be such that the needle tip 30 of a projection element 22 within a row 40 is rotationally aligned with a needle tip 30 of a projection element 22 in every other row. For example, the needle tips 30 in the first row 40a can be rotationally aligned with the needle tips 30 in a third row 40c, with the second row 40b being both between and directly adjacent to each of the first and third rows 40a, 40c.

The outer shell 16 of the tubular body 12 of the stent 10 can be formed from a thin sheet of material. According to some non-limiting examples, the outer shell 16 is formed of an elastomeric material (e.g., plastic, a polyester plastic, etc.). According to other non-limiting examples, the outer shell 16 can be formed of a metal, a polymer, or a composite. In some non-limiting examples, the outer shell 16 can be formed of rigid, thin sheets of steel, nitinol, or plastic and the “elasticity” of the material can be provided by the pattern of cuts 20. In other non-limiting examples, the outer shell 16 can be formed of soft flexible materials such as rubbers. In yet further non-limiting examples, the outer shell 16 can be formed of soluble polymers. The material of the outer shell 16 can have a shape memory, thereby allowing the projection elements 22 of the outer shell 16 to repeatedly transition between the deformed and undeformed states. According to some non-limiting examples, the outer shell 16 can define a wall thickness between about 0.01 mm and about 2 mm. According to other non-limiting examples, the wall thickness can be between about 0.05 mm and about 1 mm. According to yet further non-limiting examples, the wall thickness can be between about 0.05 mm and about 0.5 mm.

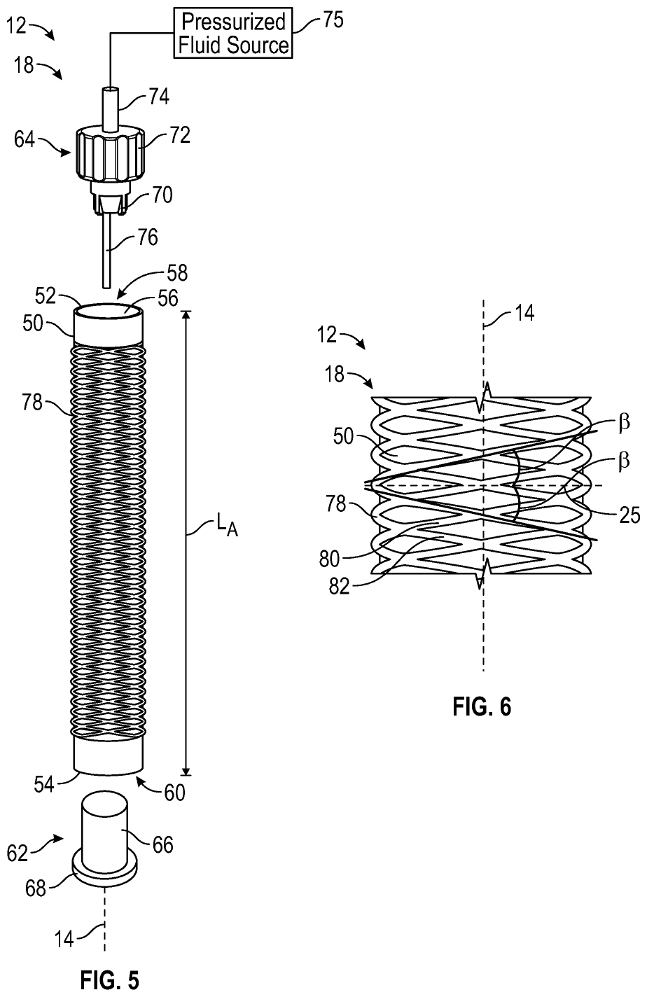

As previously described herein, the outer shell 16 of the tubular body 12 can define a lumen (e.g., a hollow core) configured to receive an actuator 18. FIG. 5 illustrates one non-limiting example of the actuator 18 configured to actuate the stent 10 between the extended and retracted positions. In the illustrated non-limiting example, the actuator 18 is a soft fluid-powered actuator (e.g., a pneumatic actuator), although other forms linear actuators are also possible. For example, the actuator can be an electric, hydraulic, mechanical, or magnetic actuator. According to other non-limiting examples, the actuator can be any form of actuator configured to provide linear motion, such as a plunger or rod manually controlled by a physician (e.g., a mechanical actuator), a piezoelectric actuator, a motor-powered actuator (e.g., a stepper motor). The actuator 18 can include a cylindrical body 50 extending along the central axis 14 from a first actuator end 52 to a second actuator end 54 opposite the first actuator end 52. The material of the cylindrical body 50 can have a shape memory, thereby allowing the cylindrical body to repeatedly transition between the extended and retracted positions. According to some non-limiting examples, the cylindrical body 50 is formed of an elastomeric material (e.g., silicone-based rubber, latex, etc.).

The body 50 of the actuator 18 can define a hollow tube including an interior cavity 56. According to some non-limiting examples, the body 50 can define a wall thickness between about 0.01 mm and about 5 mm. According to other non-limiting examples, the wall thickness can be between about 0.05 mm and about 3 mm. According to yet further non-limiting examples, the wall thickness can be between about 0.05 mm and about 2 mm.

The interior cavity 56 can extend through the body 50 between the first actuator end 52 and the second actuator end 54. In the illustrated non-limiting example, the interior cavity 56 forms a first opening 58 at the first actuator end 52 and a second opening 60 at the second actuator end 54. The actuator can also include a plug 62 and a cap 64. The plug 62 can be coupled at the second actuator end 54 of the actuator 18 to enclose the second opening 60. The plug 62 includes a plug boss 66 and a plug flange 68 at a distal end thereof extending radially outward from the plug boss 66. The plug boss can be configured to be received within the interior cavity 56 of the body 50. The plug flange 68 can be configured to abut the second actuator end 54 of the body 50, when the actuator 18 is in an assembled state (see, e.g., FIG. 2). According to some non-limiting examples, the plug 62 can define a press-fit between the plug boss 66 and the interior cavity 56 of the body 50 to form a fluid impervious seal. According to the illustrated non-limiting example, the plug 62 can be formed of an elastomeric material or a hard material (e.g., a plastic).

The cap 64 can be coupled at the first actuator end 52 of the actuator 18 to enclose the first opening 58. The body 50, plug 62, and cap 64 together define and enclose the interior cavity 56. The cap 64 can include a cap boss 70 and a cap flange 72 at a distal end thereof and extending radially outward from the cap boss 70. The cap boss 70 can be configured to be received within the first opening 58. The cap flange 72 can be configured to abut the first actuator end 52 of the body 50, when the actuator 18 is in the assembled state, to form a fluid impervious seal with the body 50. According to the illustrated non-limiting example, the cap 64 can be formed of an elastomeric material or a hard material (e.g., a plastic). According to some non-limiting examples, the cap 64 can include a nylon plastic quick-turn plug.

The cap 64 can include an inlet port 74 and a fluid passage 76 in fluid communication with the inlet port 74. The fluid passage 76 is configured to provide fluid communication between the inlet port 74 and the interior cavity of the actuator 18. The inlet port 74 can extend axially outward from the first end 21 of the outer shell 16 of the stent 10 (see FIG. 2). The inlet port 74 can be configured to be coupled to a pressurized fluid source 75 (e.g., compressed air), thereby allowing fluid from the pressurized fluid source to enter the interior cavity 56 and extend or retract the actuator 18. In the illustrated non-limiting example, the fluid passage 76 can be configured as a blunt needle (e.g., a 20G blunt needle). In the illustrated non-limiting example, the inlet port 74 can be configured as a barbed fitting.

According to some non-limiting examples, the actuator 18 can include one or more metal elements to enable tracking of the stent 10 inside the tubular structures (e.g., vasculature) of a subject during delivery, deployment, and removal using imaging, such as x-ray. For example, a steel ball (e.g., 0.5 mm in diameter) can be included in (or adjacent to) the first and second actuator ends 52, 54. The two steel balls can allow the tracking of the stent 10, in addition to the ability to confirm expansion or retraction of the actuator 18 via tracking the distance between the two steel balls. According to one non-limiting example, a first steel ball can be included (e.g., molded or cast into) the plug 62 and a second steel ball can be included in the cap 64. According to another non-limiting example, the steel balls can be included in the body 50 of the actuator 18, and axially separated from one another.

Referring now to FIGS. 5 and 6, the body 50 can include a fiber reinforcement 78 configured to constrain the deformation of the actuator 18 in the radial direction. Restricting the radial deformation can enable an increased performance in the axial direction forming an extensional actuator. The fiber reinforcement 78 can extends along at least a portion of the axial length L_{A} of the actuator 18. For example, the fiber reinforcement 78 can extend along at least 50% of the length L_{A} of the body 50. According to some non-limiting examples, the fiber reinforcement 78 can extend along between about 50% and about 100% of the length L_{A} of the body 50. According to the illustrated non-limiting example, the fiber reinforcement 78 can extend along between about 80% and about 95% of the length L_{A} of the body 50. According to some non-limiting examples, the fiber reinforcement 78 can be formed of Kevlar fibers. According to other non-limiting examples, the fiber reinforcement 78 can be formed of metal fibers. According to some non-limiting examples, the body 50 can be reinforced using rigid, circular rings along the length of the body 50 of the actuator 18. For example, a plurality of rigid (e.g., steel, nitinol, or plastic) circular rings can be arranged and axially separated along the length of the body 50 to prevent radial expansion of the body 50 and allow for axial extension.

As best illustrated in FIG. 6, the fiber reinforcement 78 can include strands of fibers arranged in a helical pattern. The fiber reinforcement 78 can include a first helical strand 80 wrapped around the body 50 in a first axial direction and a second helical strand 82 wrapped around the body 50 in a second axial direction opposite the first direction, thereby forming the helical pattern. The helical pattern can be defined by a characteristic fiber angle β, as measured when the actuator 18 is in a retracted position. The fiber angle β can be described as the angle of the wrapping of either one of the first and second strands 80, 82 relative to the plane 25 intersecting and orthogonal to the central axis 14. According to some non-limiting examples, the helical pattern can define a fiber angle β between about 1 degrees and about 60 degrees. According to other non-limiting examples, the helical pattern can define a fiber angle β between about 5 degrees and about 45 degrees. According to yet further non-limiting examples, the helical pattern can define a fiber angle β between about 5 degrees and about 30 degrees.

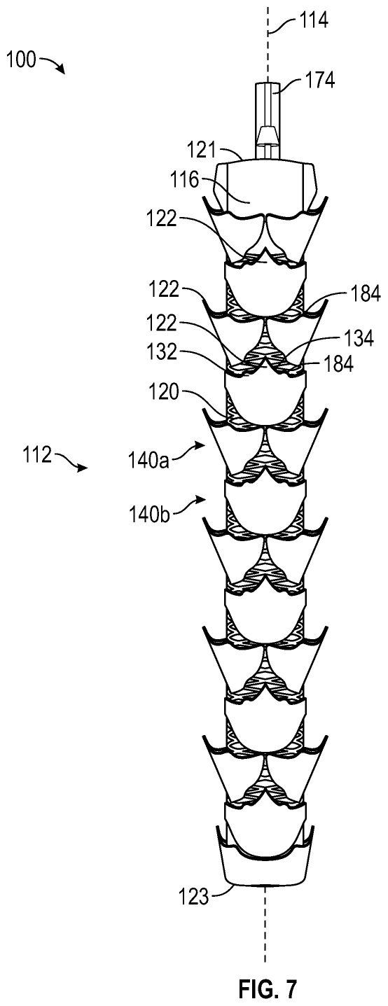

FIG. 7 illustrates another non limiting example of a stent 100. In the illustrated non-limiting example, unless otherwise described below or illustrated in the figure, like elements are labeled with like reference numerals in the 100's (e.g., projection element 22 is labeled as projection element 122). The stent 100 of FIG. 7 is substantially similar to that of the stent 10 of FIG. 1, as such, only aspects that differ from those previously described will be discussed. For example, in the illustrated non-limiting example, a first row 140a of circumferentially spaced cuts 120 is rotationally offset from a second, adjacent row 140b of circumferentially spaced cuts 120 by approximately 180 degrees.

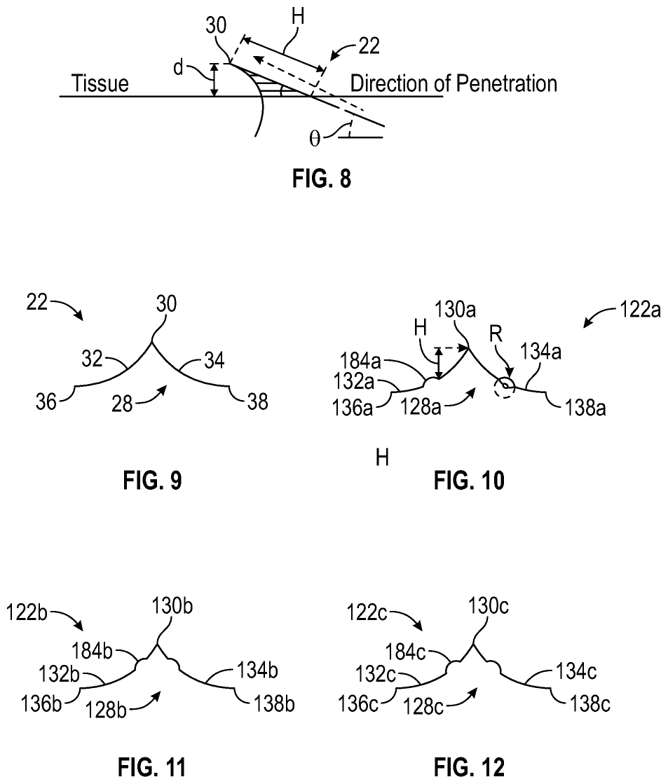

The projection elements 122 illustrated in FIG. 7 can include one or more protrusions 184 located along the first and second edges 132, 134. The protrusions 184 can be configured to control a penetration depth of the projection elements 122 (e.g., needles) into the tissue of a subject. As best illustrated in FIG. 8, the penetration depth of projection elements (e.g., projection elements 22, 122, etc.) can be characterized by the effective needle length H and the penetration depth d. The effective needle length H can be defined by a distance between the needle tip 30 of the projection element 22 and either one of a base 28 of the projection element (e.g., projection element 22 of FIG. 1) or a protrusion 184 of the projection element (e.g., projection element 122 of FIG. 7). The penetration depth d can be defined as the radial distance the needle tip of a projection element has penetrated into the tissue of a subject.

FIG. 9 illustrates one non-limiting example of a projection element 22, such as those illustrated in the stent 10 of FIGS. 1-4. In the illustrated non-limiting example, the first and second edges 32, 34 of the projection element 22 lack any protrusions. FIGS. 10-12 illustrate non-limiting examples of protrusions 184a, 184b, 184c, such as those illustrated in the stent 100 of FIG. 7, along the first and second edges 132, 134 of the projection elements 122 defining various effective needle lengths H (see FIG. 10). For example, the protrusions 184 (e.g., 184a, 184b, 184c) can be arranged at a distance away from the needle tip 130 that is between about 10% to about 95% of the total length of the projection element. According to some non-limiting examples, the protrusions 184 can be arranged at a distance away from the needle tip 130 that is between about 30% to about 95% of the total length of the projection element.

As illustrated in FIG. 10, each of the first and second edges 132a, 134a of the projection element 122a include a round, dimple-shaped protrusion 184a. The protrusion 184a can define a radius R. According to some non-limiting examples, the radius R can be between about 0.1 mm and about 5 mm. According to other non-limiting examples, the radius R can be between about 0.5 mm and about 2.5 mm.

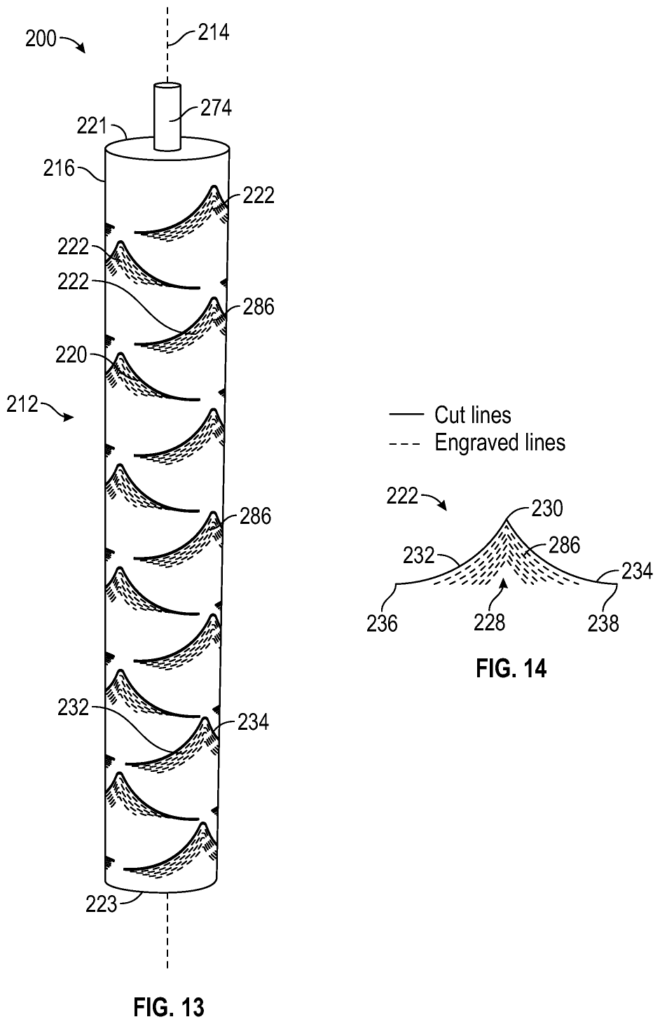

FIG. 13 illustrates another non limiting example of a stent 200. In the illustrated non-limiting example, unless otherwise described below or illustrated in the figure, like elements are labeled with like reference numerals in the 200's (e.g., projection element 22 is labeled as projection element 222). The stent 200 of FIG. 13 is substantially similar to that of the stent 10 of FIG. 1, as such, only aspects that differ from those previously described will be discussed. In the illustrated non-limiting example, the plurality of projection elements 222 can include one or more etched striations 286 (e.g., lines) formed into the outer surface of the outer shell 216.

As best illustrated in FIG. 14, the projection element 222 can include a plurality of striations 286. The striations 286 can be configured to provide a more robust surface for the loading of therapeutic agents or coatings onto the projection elements. For example, the striations 286 can improve adhesion between the surface of the outer shell 216 and a therapeutic coating or surface coating layer. The plurality of striations 286 can be shaped similar to the triangular projection element 222, such that the lines formed by the striations 286 are substantially parallel (e.g., evenly offset from) the first and second edges 232, 234 of the projection element 222. According to some non-limiting examples, the projection element 222 can include between about 1 and about 20 striations 286. According to some non-limiting examples, the projection element 222 can include between about 1 and about 10 striations 286. In the illustrated non-limiting example, the projection element 222 includes six striations 286. The striations 286 can be evenly separated (e.g., offset from) an adjacent striation. For example, the pattern of striations 286 can define a spacing between adjacent striations 286 that is between about 0.05 mm and about 2 mm. According to some non-limiting examples, the pattern of striations 286 can define a spacing between adjacent striations 286 that is between about 0.1 mm and about 1 mm.

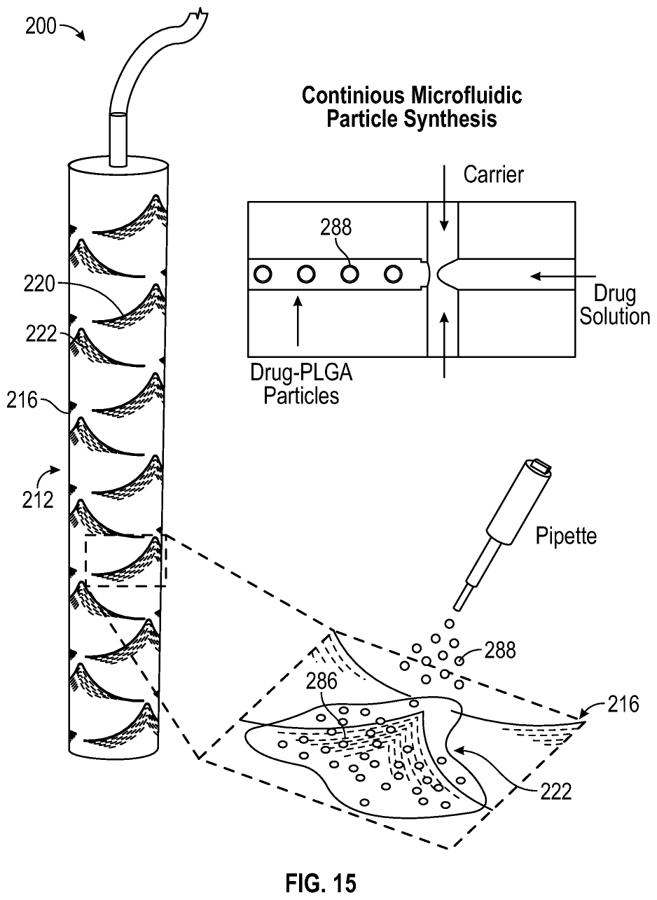

Referring now to FIG. 15, stents (e.g., stent 10, 100, 200, etc.) can be configured as drug eluting stents. For example, at least a portion of the stent can include, or be coated in, a therapeutic agent. According to one non-limiting example, the projection elements 222 of the stent 200 can include a therapeutic agent. For example, the projection elements 222 can be coated with a therapeutic agent in the form of drug particles 288 (e.g., drug-loaded polymeric particles) to enable the local delivery of therapeutics to tissues through circumferential injections within the tubular structures of a subject. According to one non-limiting example, the projection elements 222 (e.g., needles) of the stent 200 can be coated by pipetting a therapeutic agent via a pipet 290. The therapeutic agent can be entrapped or concentrated on the projection elements 222 via the striations 286 thereon. According to some non-limiting examples, the stent 200 can include polymeric sacrificial layers surrounding the stent 200 that are configured to protect the drug-coated particles, which can also increase drug loading capacity.

According to another non-limiting example, at least a portion of the stent can be formed of a soluble material that includes a therapeutic agent, such that the therapeutic agent can be released into tissues of the subject via dissolution of the soluble material. For example, the projection elements 222, or a portion thereof (e.g., the tip of the projection element), of the stent 200 can be formed of a soluble material containing the therapeutic agent. According to one non-limiting example, the stents 200 can include bi-material outer shells 216, which can include plastic hinges and soluble material projection elements 222. This can facilitate the removal of the stent 200. For example, the stent 200 can be more easily removed atraumatically once the therapeutic agent has been released into the tissues of the subject via the dissolution of the soluble projection elements 222. This can be beneficial, for example, within delicate tubular structures of a subject, such as the vasculature regions.

According to one non-limiting example, the therapeutic agent can include an anti-inflammatory drug (e.g., budesonide, prednisone, colchicine, resveratol, etc.), and anti-proliferative drugs (e.g., paclitaxel, everolimus, sirolimus, among other-limus agents, etc.), for delivery to walls of the tubular structures of the vasculature. In the illustrated embodiment, therapeutic agents can be encapsulated into poly lactic-co-glycolic acid (“PLGA”) microparticles using a continuous microfluidic droplet generation method (generally illustrated in FIG. 15). According to some non-limiting examples, the drug particles 228 can be formulated with various concentrations of the therapeutic agent. Additionally, fluorescent functionality can be added to the therapeutic agent coatings via a fluorescent agent configured to allow for confirmation of the therapeutic agent delivery using various forms of imagery.

In the above description, reference is made to various dimensions, parameters, and characteristics of the stent 10 and its components. It is to be understood that these components can be sized based on the intended application. For example, within the vasculature, stents 10 can be configured for placement within the heart, arteries, veins, etc. Dimensions and parameters of the stents 10 can be chosen based on the application or dimensions of the tubular structures of the vasculature for a given subject. For example, depending on the target position of deployment of the stent, a desired diameter and length of the stent may be determined (i.e., based on a diameter and length of the target position). Based on a determined diameter and length of the stent, the pattern of cuts 20 (e.g., needle, length, cut angle, hinge length, etc.) can be determined such that the resulting kirigami stent 10 expands to reach a desired penetration depth. For example, hinge length can be determined or calculated based on needle length, cut angle, thickness, and/or material of the outer shell 16 to provide the pop-up deployment motion of the projection elements 22. Some examples of target positions, which can influence dimensions of the stent 10, can include aortas, usually less than 3 cm in diameter, coronary arteries, usually 2-4 mm in diameter, femoro-popliteal arteries, usually 4-7 mm, and vena cava, usually 15-20 mm in diameter. The length can vary depending on the anatomy being targeted (e.g., coronary lesions are typically shorter than femoro-popliteal).

### Methods of Inserting/Removing a Kirigami-Inspired Stent

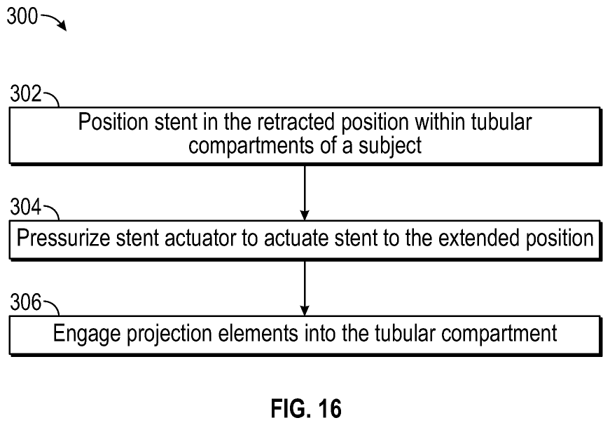

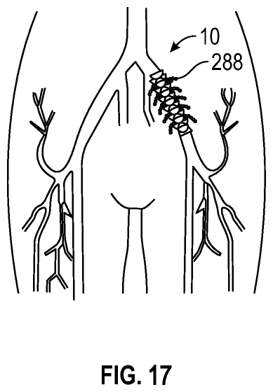

Referring now to FIGS. 1, 2, and 16, the kirigami-inspired stents 10 are capable of reversible shape transformation from a retracted position (FIG. 2), in which the projection elements 22 are in a flat, undeformed state resulting in a smooth outer surface of the outer shell 16, to an extended position (FIG. 1), in which the projection elements 22 are transitioned into a deformed state and configured to provide popped-up needles configured for injections into a tissue of a subject. With the tubular body 12 of the stent 10 in the retracted position, the stent 10 can be delivered and removed from tubular structures within the subject (e.g., arteries and other vasculature structures). With the tubular body 12 of the stent 10 in the extended position, the projection elements 22 (e.g., needles) of the stent 10 can deliver circumferential injections to pierce the tissue of the subject, and according to some non-limiting examples, deliver a therapeutic agent into the injection sites. Thus, the stent systems described herein can provide facile, in vivo delivery, robust deployment, and safe removal of a stent configured for injections, and according to some non-limiting examples, providing a drug releasing system. It is to be understood that the following method 300 can be applied to each of the stents described herein (e.g., stent 10, 100, 200). In the following description reference will be made to the stent 10 of FIGS. 1-4.

The method can begin at 302 by inserting the stent 10 into a tubular tissue structure of a subject in a first, insertion direction (e.g., relative to the central axis 14). For example, the stent 10 can be inserted to a target tissue site in the vasculature (FIG. 17), as well as valvular regions, myocardial, and endocardial regions, by applying a pushing force to the first end of the tubular body 12 of the stent 10. During insertion, the stent 10 is in the retracted position (FIG. 2) with the actuator 18 unpressurized.

Once the stent 10 is positioned at the tissue site of interest, the actuator 18 can be actuated 304 from the retracted position towards the extended position, thereby deploying the projection elements 22 radially outward into the deformed state. For example, the actuator 18 can be pressurized by the pressurized fluid source 75 coupled to the inlet port 74 and the actuator 18 can begin to elongate to engage the enclosed first and second ends 21, 23 of the outer shell 16 of the tubular body 12, thereby elongating the outer shell 16 and deforming the projection elements 22 to deploy radially outwards.

With the stent 10 in the extended position, the projection elements 22 can engage 306 the tissue of the subject to form a pattern of circumferential injection sites into the tissue. According to some non-limiting examples, the stent 10 can be moved in a second, removal direction by applying a pulling force to the first end of the tubular body 12 of the stent 10. By moving the stent 10 in the second direction with the projection elements 22 deployed, the projection elements can be further driven into the tissue of the subject to increase the insertion depth of the projection elements 22. For example, the projection elements 22, when deployed, generally extend from the second end 23 towards the first end 21 of the tubular body 12, owing to the needle angle θ (see, e.g., FIG. 3). Thus, movement of the stent 10 in the second direction (towards the first end 21) can drive the projection elements 22 into the tissue of the subject.

As previously described, the projection elements 22 can be loaded with a therapeutic agent (see, e.g., FIG. 9), and insertion of the projection elements 22 can be configured to deposit the therapeutic agent (e.g., in the form of drug particles 288) at the circumferential injection sites. According to some non-limiting examples, the stent can be left in place for a period of minutes, hours, or days (e.g., up to a week or more) to provide prolonged delivery of the therapeutic agent via the drug-loaded projection elements 22. Thus, the stents 10 described herein can provide delivery of therapeutic agents to vascular and valvular regions, as well as endocardial and myocardial regions, via insertion of the stent 10 and the deployment of the projection elements 22 thereon.

For removal of the stent 10, the stent 10 can be moved in the first direction (towards the second end 23) to remove the projection elements 22 from the tissue of the subject. With the projection elements 22 removed, the stent 10 can be actuated from the extended position towards the retracted position to stow the projection elements into the undeformed state. Once the stent 10 is in the retracted position, the stent 10 can be removed from the subject by moving the stent 10 in the second, removal direction, for example, by again applying a pulling force to the first end of the tubular body 12 of the stent 10.

According to some non-limiting examples, a tube dimensioned to receive the stent 10 therein can be inserted into the tubular structure of the subject prior to insertion of the stent 10. The tube can be configured to guide delivery of the stent 10 to a tissue site of interest. For example, a first sheath of a first size (e.g., 20 F, 22 F, etc.) can be percutaneously placed in the tubular structure of the subject (e.g., an artery) under the guidance of imagery (e.g., ultrasound and fluoroscopic guidance). With the first sheath placed, the stent 10 can be pre-loaded into a second sheath of a second size smaller than the first size (e.g., 18 F). The second sheath can then be inserted through the previously placed first sheath positioned in the tubular structure of the subject (e.g., a sheath-in-sheath approach). The stent 10 can then be unsheathed in the tubular structure. After being unsheathed, steps 302-306 of the method 300 can be followed to allow the stent 10 to engage the walls of the tubular structure.

According to some non-limiting examples, an occluding balloon can be arranged upstream or downstream of the target site. For example, a third sheath (e.g., a 12 F vascular introducer sheath) can be placed and an occluding balloon can be inserted and inflated to occlude flow of fluids through the tubular structure of the subject prior-to and while the stent is being placed.

### Methods of Making a Kirigami-Inspired Stent

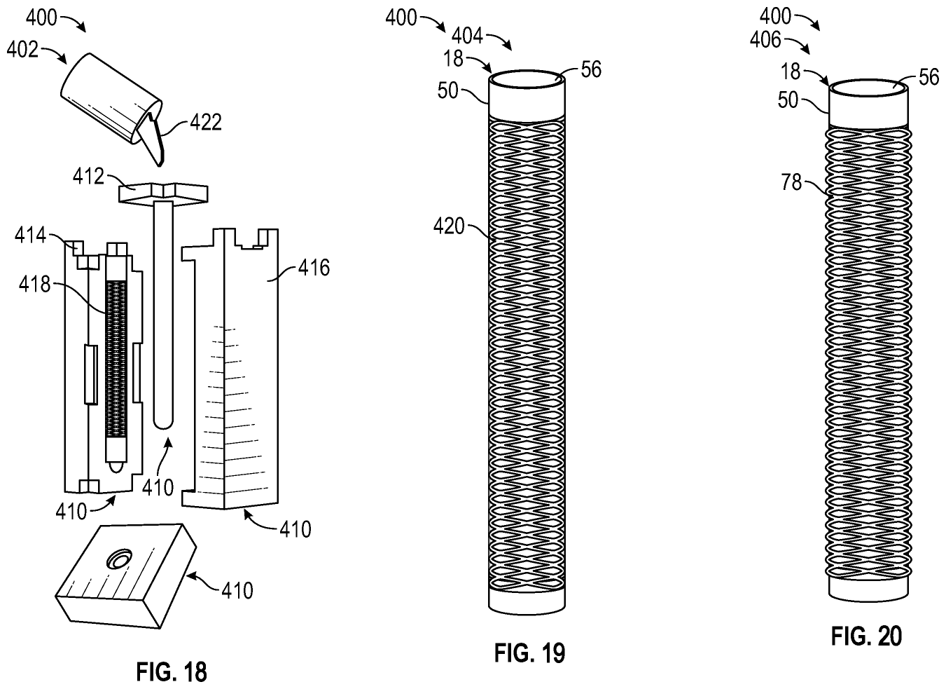

Referring now to FIGS. 18-20, a non-limiting example of a method 400 of making the actuator 18 for the stent 10 is illustrated. It is to be understood that the following method 400 can be applied to each of the stents described herein (e.g., stent 10, 100, 200). In the following description reference will be made to the stent 10 of FIGS. 1-4. As illustrated in FIG. 18, the body 50 of the actuator 18 can be formed via a casting or injection molding process 402. The casting or injection molding process can include providing a multi-piece mold 410 (FIG. 18), including a first part 412 forming the interior cavity 56, and second and third parts 414, 416 forming the body 50. In the illustrated non-limiting example, the second and third parts 414, 416 of the mold 410 can include a pattern of helical protrusions 418 configured to form helical recesses 420 along the body 50 to receive the fiber reinforcement 78 (FIG. 19).

According to some non-limiting examples, the mold 410 can be sprayed with a releasing agent for easy demolding. Then, the elastomeric actuator body 50 and plug 62 can be cast separately using an elastomeric material (e.g., a silicone-base rubber, vinylpolysiloxane, a-silicone). According to some non-limiting examples, the elastomeric material can be a duplicating elastomer (e.g., Elite Double 8). The casted mixture can be mixed for a predetermined period of time (e.g., two minutes), placed in a vacuum for degassing, and then allowed to set at a predetermined temperature (e.g., room temperature) for a predetermined period of time (e.g., thirty minutes) to cure.

With the body 50 formed, strands of fiber reinforcement material can be wrapped 404, 406 within the helical recesses 420 along the body 50 (FIG. 20) to form the helical-patterned fiber reinforcement 78. According to some non-limiting examples, a uniform thin layer of a silicone adhesive can be applied to the outer surface of the fiber-reinforced actuator body 50 to enhance the bonding between the fiber and elastomer. The extensional actuator body 50 can then be left to cure at a predetermined temperature for a predetermined period of time (e.g., room temperature for 30 min), allowing the silicone adhesive to dry. Then, the plug 62 and the cap 64 can be coupled with the body 50 (e.g., via an adhesive) to seal the interior cavity 56 (see FIG. 5).

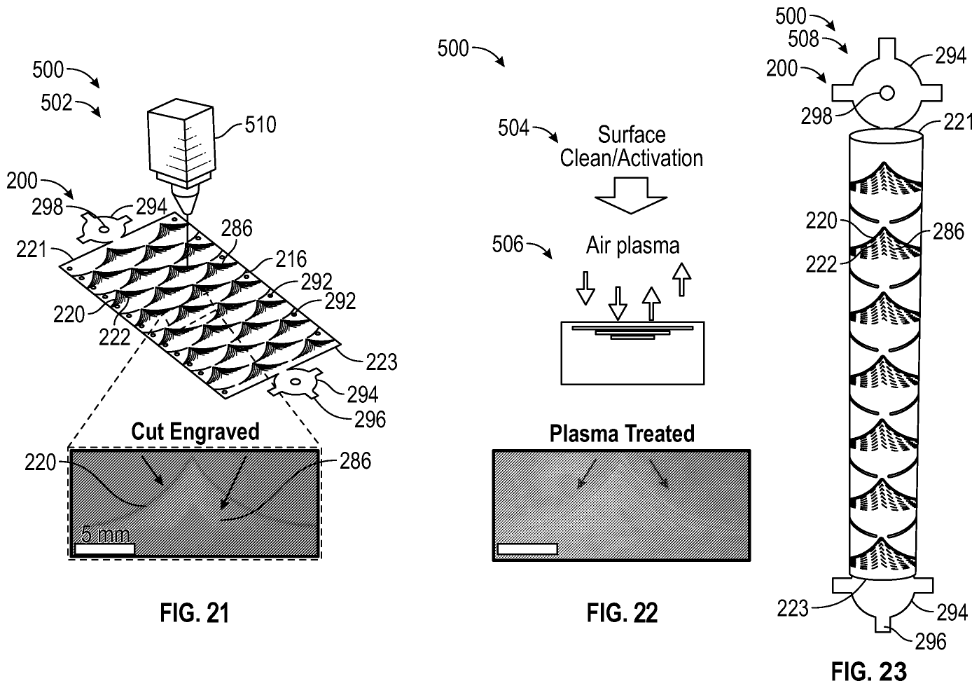

Referring now to FIGS. 21-23, a non-limiting example of a method 500 of making the outer shell 216 for the stent 200 is illustrated. It is to be understood that the following method 500 can be applied to each of the stents described herein (e.g., stent 10, 100, 200). In the following description reference will be made to the stent 200 of FIGS. 13-14. As illustrated in FIG. 21, the stent 200 can be cut 502 from a flat sheet of material, and then later formed into a cylindrical shell 508. In the illustrated non-limiting example, the cuts 220 were formed via a laser cutter 510 (e.g., a CO₂ laser). In the specific illustrated non-limiting example, the stent 200 is composed of a periodic array of 2×13 projection elements 222 (e.g., 26 projection elements). Although, other configurations of arrays and total number of projection elements are also envisioned. As illustrated in FIG. 21, the laser cutter 510 can also form the etched striations 286 on the outer surface of the outer shell 216. For example, the laser cutter 510 can form the cuts 220 at a first power and the striations 286 can be formed at a second power that is lower than the first power.

According to some non-limiting examples, the outer shell 216 can include small apertures 292 perforated along lateral edges of the outer shell 216, which can be used to facilitate alignment when formed into a cylindrical shape. According to the illustrated non-limiting example, circular cutouts 294 can be coupled to the first and second ends 221, 223 of the outer shell 216. The circular cutouts 294 can be configured as end caps for the outer shell 216 when formed into a cylindrical shape. In the illustrated non-limiting example, the circular cutouts 294 can include one or more tabs 296 extending outward from the circular cutouts 294. The tabs 296 can be configured to be coupled to the first and second ends 221, 223 of the outer shell 216 (e.g., via an adhesive) to secure the circular cutouts 294 to the outer shell 216. The circular cutout 294 arranged at the first end 221 of the outer shell 216 can include a central aperture 298. The central aperture 298 can be configured to receive the inlet port 274 (see FIG. 13) such that the inlet port 274 can extend axially away from the outer shell 216 through the first end 221 thereof.

As illustrated in FIG. 22, some surfaces (e.g., such as plastic surfaces or surfaces comprised of elastomeric materials) can be hydrophobic, which can lead to incompatibility with surface coatings, such as therapeutic agent coatings. To increase the adhesion bond to surface coatings, an air plasma treatment 504, 506 can be utilized to micro clean and alter the surface properties of the kirigami surfaces for adhesion improvement. According to one non-limiting example, the surfaces of the outer shell 216 can be treated in air plasma 506 with high radio frequency for a predetermined period of time (e.g., at 500 mTorr for 1 hour) using a plasma cleaner device (e.g., a high power expanded cleaner). The plasma treatment results in the creation of hydrophilic surfaces of the outer shell 216 and improvement in the adhesive bond created between the outer shell 216 and surface coatings, such as therapeutic agent coatings like a drug-coated film, that can facilitate the drug solution coating and enhance the drug film stability.

According to some non-limiting example, a surface coating can include a radiopaque coating. For example, at least a portion of the outer shell 216 can be coated in a radiopaque coating. The radiopaque coating can make the outer shell 216 of the stent 200 radiopaque. According to some non-limiting examples, the entire outer shell 216 can be coated with the radiopaque coating. According to other non-limiting examples, at least the projection elements 222 can be coated with the radiopaque coating. According to some non-limiting examples, the outer shell 216 can be coated with a thin layer of tungsten filled conductive ink (e.g., RO-948 Radio Opaque Ink, MICROCHEM). According to other non-limiting examples, radiopaque markers can be arranged on opposing ends of the device.

Finally, as illustrated in FIG. 23, the outer shell 216 can be formed into a cylindrical-shaped shell and the lateral edges can be coupled together (e.g., via an adhesive) with the outer surface with the striations 286 facing outward. In this configuration, the outer shell 216 can then receive an actuator (e.g., actuator 18, FIG. 5). Once the actuator 18 is within the outer shell 216, the circular cutouts 294 can be coupled to enclose the first and second ends 221, 223 (e.g., via an adhesive) (see, e.g., FIG. 13).

### Examples

The following description includes particular non-limiting examples of stents that utilize the systems and methods previously described herein. The following examples are not intended to limit the disclosure. In the following description, the mechanics of kirigami stents for injection and deposit fluorescent polymeric particles are characterized. These systems were evaluated in vivo in pigs. Such systems can be applied for a range of diseases affecting the cardiovascular system such as aortic stenosis and atherosclerotic cardiovascular diseases.

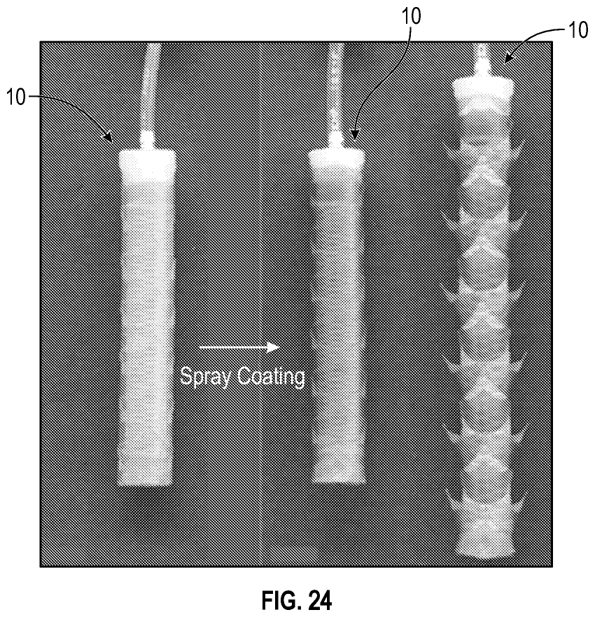

FIG. 24 illustrates experimental images of the endovascular stent 10 in an undeformed (center) and deployed (right) configurations. To enable loading and delivery of polymeric particles with the injectable stent system, the external surface of the stent 10 of FIG. 24 was coated with a solution of fluorescent magnetic polystyrene microparticles.

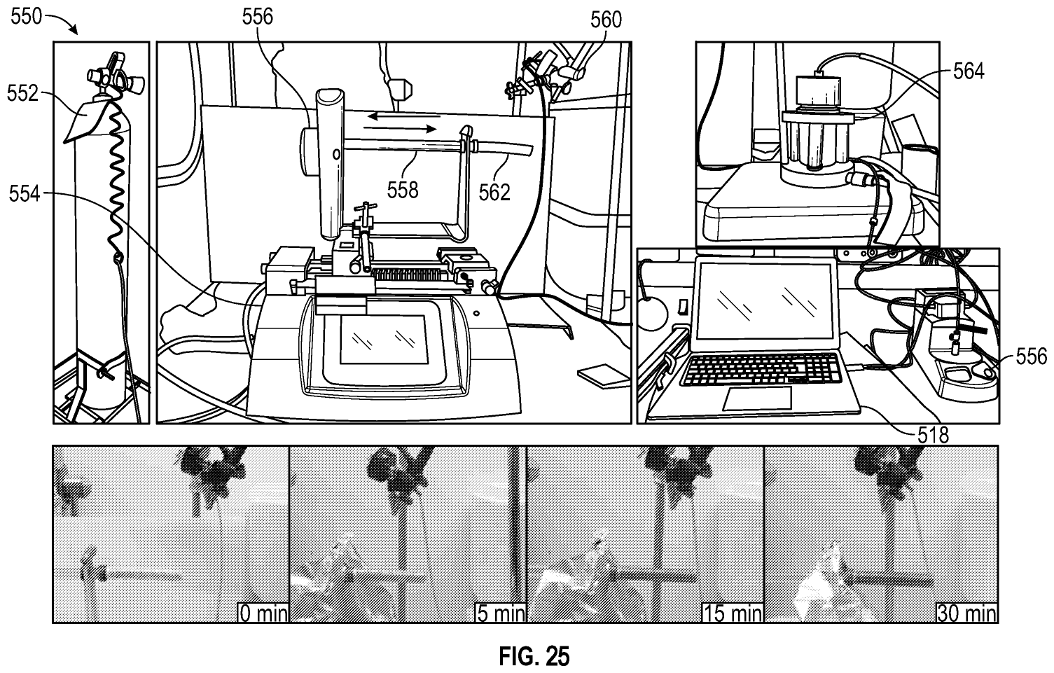

As illustrated in FIG. 25, a custom-built benchtop spray coating set-up with programmable stent movement and rotation was used to achieve a uniform thin film coating of the solution onto the kirigami stent shell. In the illustrated example, an airbrush controlled by a micro-fluidic pump and flow sensor was used to spray-coat the kirigami stent prototypes with fluorescent particle solution. The set-up 550 includes: nitrogen gas tank 552, standard infusion syringe pump 554, 20 rpm rotary fixture 556, 3D printed rotary shaft 558, airbrush 560, kirigami stent prototype 562 (e.g., stent 10, 100, 200), pressurized vessel containing the coating solution 564, micro-fluidic pump with a flow sensor 566, and PC controlling unit 568. The snapshots of the coating process at different time points (0, 5, 15, and 30 min) are illustrated in the bottom row of FIG. 25. One end of the shaft 558 was connected to a 20 rpm rotary fixture 556, while the other end held the stent prototype 562. The rotary fixture 556 was secured to a syringe pump 554 head, which provided a linear motion with 15 ml/min infuse or withdraw rate for a 50 ml target volume per coating step. This resulted in forward and backward motion (corresponding to infuse and withdraw steps) of the stent 562 with 24 mm/min speed for 8 cm displacement under a fixed airbrush 560, while the stent 562 rotates during the whole coating process. Such a rotation and linear motion ensure that the whole stent is covered with a uniform coating layer.

The airbrush 560—used to spray the coating solution through its nozzle—was connected with a silicone tubing to a 30 ml pressurized coating solution vessel 564 and placed on a magnetic stirrer for continuous mixing, feeding and spraying the solution. The vessel 564 was equipped with a pressure pump 566 controlled by software (e.g., on the PC controlling unit 568). Two nitrogen gas tanks 552 were used to supply pressure for the pressure pump 566 (400 KPa) and airbrush 560 (50 KPa) during the coating process. The feeding pressure was optimized (5-60 KPa) and set to 40 KPa (equal to 40 μl/min) to reach a constant solution flow and uniform spraying pattern. The whole coating process consisted of eight coating steps (four infuse and four withdraw).

In vivo sustained drug release through deposition of polymeric particles loaded with therapeutics

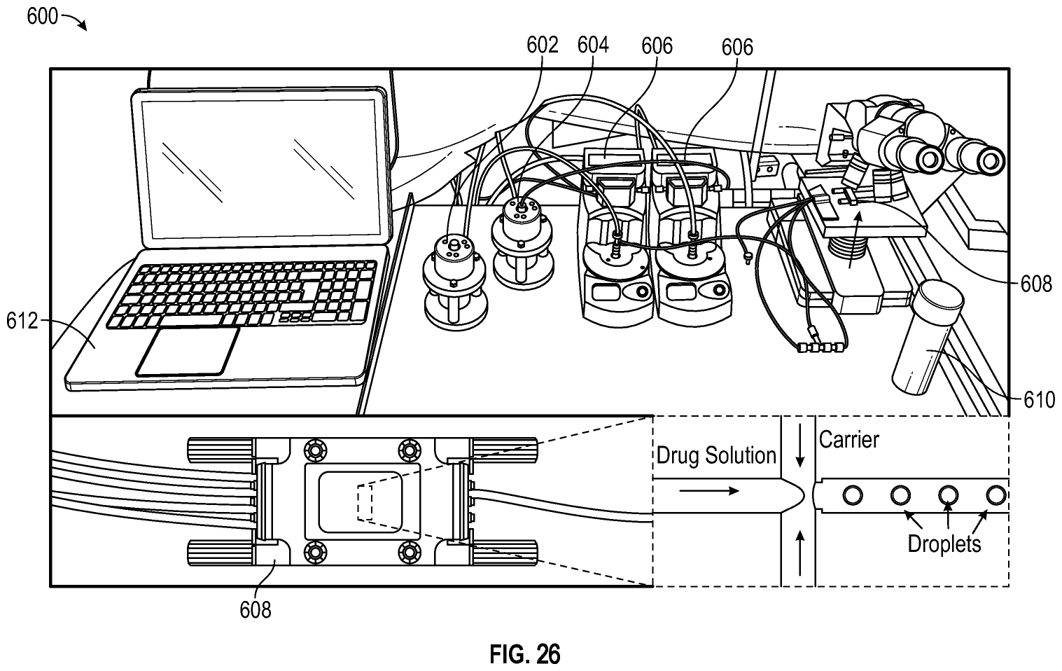

The injectable stent can deliver drug depots for up to a week through multipoint submucosal deposition of drug-loaded polymeric particles. For example, budesonide, an anti-inflammatory drug was encapsulated into poly lactic-co-glycolic acid (“PLGA”) microparticles using continuous microfluidic droplet generation method. Budesonide-PLGA [Poly(D,L-lactide-co-glycolide) ester terminated, lactide:glycolide 75:25, Mw 76,000-115,000, Sigma Aldrich] microparticles were synthesized using a continuous microfluidic drug-PLGA droplet generation method, shown in FIG. 26.

The set-up 600 includes: pressurized vessel 602 containing the Water/PVA mixture as aqueous stream, 30 ml pressurized vessel 604 containing budesonide and PLGA dissolved in DCM, pressure pumps 606 equipped with flow rate sensors for transferring aqueous and organic phases to the chip 608, one reagent 100 μm hydrophilic glass 3D flow-focusing microfluidic glass chip 608 and customized holder—see the magnified view of the channel configuration in the chip 608 in the bottom-left of FIG. 26, Siliconized glass stirred vessel 610 for collecting synthesized microparticles and solvent evaporation, and PC with software 612 for controlling pumps 606 with digital microscope interface for viewing and monitoring the droplet formation process.

The one reagent glass 3D flow-focusing microfluidic chip 608 with hydrophilic surface and 100 μm deep channels was used, followed by a solvent extraction step. Two partially miscible solvents including dichloromethane and water were used as drug solvent/carrier and droplets carrier phases, respectively. Budesonide (75,100, and 125 mg/ml) and 1% w/v PLGA were dissolved in DCM as an organic fluid. 2% w/v PVA in double-distilled water was used as an aqueous/carrier phase for droplet generation. All fluids passed through a 0.2 μm pore microfilter before droplet production. To generate fluorescence-sensitive budesonide-PLGA particles, 0.3% w/v of PLGA-SH (LG 50:50, PolySciTech) and 20 μl of Alexa Flour 647 C2 Maleimide dye (Invitrogen) was also added to the budesonide-PLGA solution.

The microfluidic system set-up 600 includes two pressure pumps 606 equipped with in-line flow rate sensors to monitor and control the streams flow rates. Two flow rate sensors, 30-1000 μl/min and 1-50 μl/min, were employed in the organic line and aqueous line, respectively. An air compressor (not shown) provided the supply pressure for the pressure pumps 606 at 400 KPa working pressure. The pumps 606 were connected to 30/400 ml and 30 ml volume remote pressure chambers 602, 604 placed on magnetic stirrer for continuous mixing and delivering of PVA in water and DCM-PLGA-Budesonide solution to the chip 608 with 10 μl/min aqueous/carrier rate and 1.35 μl/min organic/drug-PLGA solutions rate, respectively. The particle synthesis process was continuously continued to reach 500 mg of particles while the DCM solvent was evaporating/by connecting the particle's collection siliconized stirred vessel to very mild vacuum pressure (about 650 Torr). Three formulations of budesonide-PLGA particles was synthesized with 75,100, and 125 mg/ml concentration of budesonide, denoted by BUD75, BUD100, and BUD125, respectively. Additionally, 100 mg/ml concentration of fluorescent budesonide-PLGA particles (BUD 100 F) was synthesized via addition of Alexa Flour 647 C2 Maleimide as described.

In vivo delivery, deployment, and removal of endovascular stents

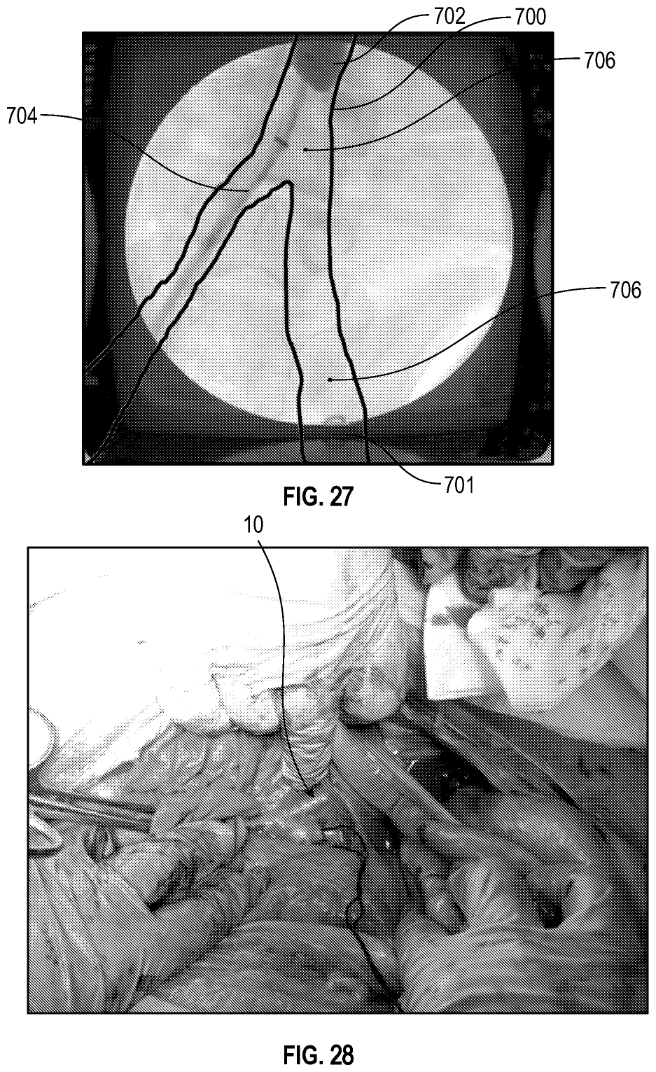

As illustrated in FIGS. 27 and 28, the kirigami stents were deployed for in vivo evaluations in a large animal model (50 to 80 kg female Yorkshire pigs ranging between 4-6 months of age). A 20 or 22 F vascular introduced sheath 701 was percutaneously placed in the femoral artery of the pig under ultrasound and fluoroscopic guidance. In the contralateral femoral artery, a 12 F vascular introducer sheath 704 was placed and a 32 mm balloon 703 was positioned in the descending aorta 700 via the 12 F sheath 704. After balloon occlusion of the descending aorta 700 with the balloon 702, the kirigami stent with 8 cm length and 5.5 mm diameter and coated with tissue marking dye, and radiopaque markers 706 on distal ends thereof, was pre-loaded into a 18 F vascular introducer that was then inserted through the previously placed 20 F sheath 701 positioned in the iliac artery 700 (“sheath-in-sheath” approach) (FIG. 27). The kirigami stent device was then unsheathed in the iliac artery 700, inflated, and was gently retracted to engage the arterial wall.

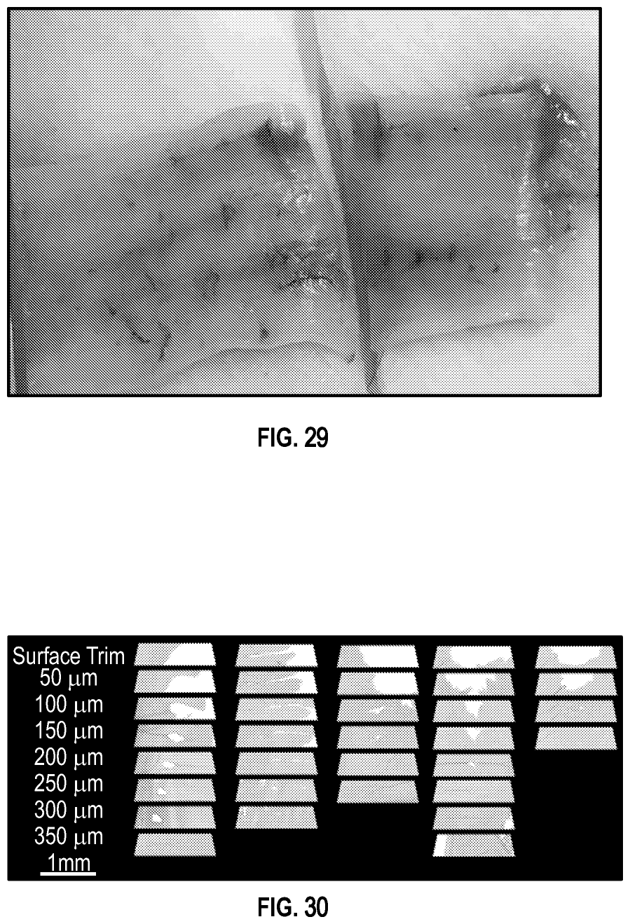

FIG. 29 illustrates a photograph of the iliac artery exposed to demonstrate the position of the coated kirigami stent. Inspection of the treated iliac artery demonstrated the presence of tissue dye marking on the luminal surface of the artery consistent with the repeated pattern of the kirigami skin which was confirmed on histologic analysis (see FIG. 30).

While various spatial and directional terms, such as top, bottom, lower, mid, lateral, horizontal, vertical, front, and the like may be used to describe examples of the present disclosure, it is understood that such terms are merely used with respect to the orientations shown in the drawings. The orientations may be inverted, rotated, or otherwise changed, such that an upper portion is a lower portion, and vice versa, horizontal becomes vertical, and the like.

Within this specification, embodiments have been described in a way which enables a clear and concise specification to be written, but it is intended and will be appreciated that embodiments may be variously combined or separated without parting from the invention. For example, it will be appreciated that all preferred features described herein are applicable to all aspects of the invention described herein.

Thus, while the invention has been described in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

Various features and advantages of the invention are set forth in the following claims.

1. A stent for treating tissue within tubular structures of a subject, the stent comprising:
a tubular body extending along a central axis and configured to move between a retracted position and an elongated position; and
a plurality of projections formed into the tubular body, each projection forming a cutting edge to pierce a tissue within a tubular structure of the subject,
wherein each projection among the plurality of projections is configured to change orientation relative to the central axis when the tubular body moves between the retracted position and the elongated position.

2. The stent of claim 1, wherein when the tubular body is in the retracted position, the plurality of projections form a cylindrical outer surface of the tubular body, and
wherein when the tubular body is in the elongated position, the plurality of projections extends radially outward from the tubular body into a deployed position to pierce the tissue proximate to the tubular body.

3. The stent of claim 1, wherein the projections define a needle angle between about 1 degree and about 90 degrees relative to the central axis when the projections are in a deployed position.

4. The stent of claim 1, wherein the projections are triangular-shaped, with a first edge and a second edge defining the cutting edge, and a base of the triangular-shaped projections defining an uncut portion of the tubular body.

5. The stent of claim 1, wherein the elongated position of the tubular body defines an elongated length that is between about 1% and about 100% greater than an initial length of the tubular body in the retracted position.

6. The stent of claim 1, wherein the plurality of projections are formed by a pattern of interconnected cuts into the tubular body.

7. The stent of claim 1, wherein the plurality of projections are circumferentially arranged around the tubular body.

8. The stent of claim 1, wherein at least a portion of the stent is coated with a therapeutic agent.

9. A stent system for treating a tissue of a subject, the system comprising:
a tubular body extending along a central axis to form a lumen within the tubular body,
an actuator received within the lumen and configured to move the tubular body between a retracted position and an elongated position; and
a pattern of a plurality of cuts formed along the tubular body and extending through the tubular body to the lumen,
wherein the pattern of the plurality of cuts deploy into a plurality of interconnected projections that are configured to extend radially away from the tubular body relative to the central axis to engage a tissue within a tubular structure of a subject when the tubular body is moved towards the elongated position.

10. The stent of claim 9, wherein when the tubular body is in the retracted position, the plurality of projections form a cylindrical outer surface of the tubular body.

11. The stent of claim 9, wherein the projections define a needle angle between about 1 degree and about 90 degrees relative to the central axis when the projection is in a deployed position.

12. The stent of claim 9, wherein the projections are triangular-shaped, with a first edge and a second edge defining a cutting edge, and a base of the triangular-shaped projections defining an uncut portion of the tubular body.

13. The stent of claim 9, wherein the actuator is configured to elongate the tubular body to an elongated length that is between about 1% and about 100% greater than an initial length of the tubular body in the retracted position.

14. The stent of claim 9, wherein the actuator is a pneumatic actuator including an actuator body having an interior cavity and an inlet port, wherein the inlet port is configured to be in fluid communication with a pressurized fluid source to provide pressurized fluid to the interior cavity.

15. The stent of claim 14, wherein the pneumatic actuator comprises an elastomeric material.

16. The stent of claim 14, wherein the actuator body includes a fiber reinforcement extending along at least a portion of the length of the actuator body.

17. The stent of claim 16, wherein the fiber reinforcement includes strands of fibers arranged in a helical pattern.

18. A method of inserting a stent into a tubular structure a subject, the method comprising:
positioning a stent to a target tissue site, the stent having a tubular body extending along a central axis to form a lumen within the tubular body,
pressurizing an actuator received within the lumen to move the tubular body from a retracted position to an elongated position,
wherein a surface of the tubular body includes a pattern of a plurality of cuts configured to deploy into a plurality of interconnected projections as the tubular body is moved into the elongated position to engage the target tissue site of the subject.

19. The method of claim 18, wherein the projections include a therapeutic agent such that the therapeutic agent is delivered to the subject when the projections engage the target tissue site.

20. The method of claim 19, wherein the stent is inserted in a first, insertion direction, and upon moving the tubular body into the elongated position, moving the stent in a second direction opposite the first direction to drive the projections into the target tissue site.
